(19) United States
(12) Patent Application Publication
(10) Pub. No.: US 2019/0099441 A1
(43) Pub. Date: Apr. 4, 2019

(54) OPHTHALMOLOGICAL COMPOSITION

(71) Applicant: Ursapharm Arzneimittel GmbH, Saarbrücken (DE)

(72) Inventors: Markus Mahler, Völklingen (DE); Isabella Warda, Saarbrücken St. Johann (DE); Ute Steinfeld, St. Ingbert (DE); Frank Holzer, St. Ingbert (DE)

(21) Appl. No.: 16/082,791

(22) PCT Filed: Mar. 7, 2017
(86) PCT No.: PCT/EP2017/055338

§ 371 Date: Sep. 6, 2018

(30) Foreign Application Priority Data
Mar. 7, 2016 (DE) 10 2016 203 696.3

(51) Int. Cl. A61K 31/728; A61K 31/505; A61K 9/00; A61P 27/04

(52) U.S. Cl. CPC A61K 31/728; A61K 31/505; A61K 47/186; A61P 27/04; A61K 9/0048

(57) ABSTRACT

The present invention relates to an ophthalmological composition with high viscosity. The composition according to the invention comprises or thereby consists of hyaluronic acid or a hyaluronic acid derivative, such as for example an ophthalmologically acceptable salt of hyaluronic acid and also ectoin or an ophthalmologically acceptable ectoin derivative. The composition is further characterised in that it comprises no further pharmaceutically active ingredient.

The present invention relates to an ophthalmological composition with high viscosity. The composition according to the invention thereby comprises or consists of hyaluronic acid or a hyaluronic acid derivative, such as for example an ophthalmologically acceptable salt of hyaluronic acid and also ectoin or an ophthalmologically acceptable ectoin derivative. The composition is further characterised in that it comprises no further pharmaceutically active ingredient.

Burning, itchy and watery eyes, having the feeling of sand in the eyes or dry eyes are symptoms of an irritated eye. Frequently, this is a sign of the eye not being supplied sufficiently with moisture. Another cause can however also be oversensitivities relative to specific substances (e.g. pollen, animal hair or house dust), so-called allergies. Since an allergy is accompanied by the same complaints, the differentiation between dry eyes and irritated eyes caused by an allergy is difficult.

Already known solutions for counteracting irritated eyes are directed generally only to one of the mentioned causes of eye irritation or eye inflammation, i.e. act either against dryness-caused or allergically-caused irritations and inflammations of the eye.

Ectoin is a natural substance which is obtained from microorganisms which live in extreme environments (e.g. salt lakes). These microorganisms form the natural substance ectoin in order to protect themselves from the extreme environmental factors prevailing there.

It is known from EP 0 671 161 B1 that ectoin and its derivatives can be used as moisturiser in cosmetic products for increasing the moisture content of the skin. EP 2 214 658 B1 describes the use of ectoin in osmolyte-containing preparations for application in the case of dry mucous membranes of the nose. Use of ectoin in solutions for preventing and treating an irritation and/or inflammation of the eye is not described in EP 0 671 161 B1.

DE 10 2014 007 423 A1 discloses, in contrast, compositions for treating inflammations in the eye which comprise ectoin. In the case of an examination, of the random sample survey type, of 59 patients, it is shown that the treatment of kerotoconjunctivitis sicca with a composition which comprises ectoin and/or hydroxyectoin and/or corresponding derivatives of these substances is somewhat more effective than treatment with a hyaluronic acid solution.

It is now the object of the present invention to indicate a solution for preventing and treating or for using in prevention and treatment of an irritation and/or inflammation of the eye, the solution being intended to be suitable both for preventing and treating a dryness-caused- and an allergically-caused irritation and/or inflammation of the eye, as a result of which a differentiation of the various causes of the symptoms is no longer required. In addition, the solution should comprise no components which lead generally to irritation of the eye or to impairment in visual capacity. The solution should thereby have as high viscosity as possible in order to ensure that the composition remains adhering well and for a long time on the surface of the eye. In addition, the solution should have as high a water-binding capacity as possible, which ensures sustained moistening of the eye.

Starting from the state of the art, it is likewise the object of the present invention to indicate an ectoin-containing ophthalmological composition which has an improved effect in the treatment of irritations and/or inflammations of the eye and can be produced more economically than conventional hyaluronic acid-containing ophthalmological compositions.

This object is achieved by the ophthalmological composition according to the features of patent claim 1. The dependent patent claims thereby represent advantageous developments.

The present invention hence relates to an ophthalmological composition, comprising or consisting of
0.055 to 2.00% by weight of hyaluronic acid and/or an ophthalmologically acceptable salt of hyaluronic acid,
0.60 to 5.00% by weight of ectoin or an ophthalmologically acceptable ectoin derivative, and also
ad 100% by weight of water,
   the composition comprising no further pharmaceutically active ingredient.

The solution according to the invention moistens cornea and conjunctiva and protects from excessive evaporation of tears. This stabilisation of the tear film soothes eye irritations which are associated with inflammatory symptoms, or which are caused by allergies. The burning and itchy feeling of the eyes disappears.

Sodium hyaluronate is a natural substance which can be found in the eye but also in other body parts. It ensures that a uniform, stable and particularly long-term-adhering moisture film is formed on the surface of the eye which cannot be rinsed off rapidly.

Ectoin increases the water binding to the cells of the eye surface and hence forms a physiological barrier on the conjunctiva, e.g. for allergy-causing substances. At the same time, ectoin stabilises the fat-soluble proportion of the tear film which protects from excessive evaporation of tear fluid.

Surprisingly, with the composition according to the invention a synergistic effect of hyaluronic acid or derivatives derived herefrom and also ectoin on the viscosity could be established. If these components are used in the concentrations according to the invention, then it is observed that the composition has a higher viscosity than the individual solutions which comprise merely one of the mentioned components (hyaluronic acid or ectoin). The viscosity of the composition according to the invention is even higher than the sum of the viscosities of the individual solutions.

The same applies for the water-binding capacity: it was shown surprisingly that, in addition to the synergistic increase in viscosity, the result is also a synergistic increase in the water-binding capacity by combination of both substances. This enables a better and more intensive moistening of the eye and stabilisation of the tear film relative to comparable eye drop formulations. With minimal raw material use, a maximum effect can be achieved by the synergistic cooperation.

These findings also have a positive effect on the production costs of the composition. Both hyaluronic acid and ectoin are expensive raw materials. The synergistic viscosity- and water-binding capacity increase proves hence to be very advantageous for saving on raw materials and costs. As a result of the synergistic effect, a selected viscosity and water-binding capacity can be produced with a lower raw material use than in the case of the ophthalmological composition from the state of the art, as a result of which cost savings can be made.

The composition according to the invention which comprises both ectoin and/or an ectoin derivative and hyaluronic acid and/or a salt of hyaluronic acid in the mentioned concentrations adheres better, during application, to the cornea and to the eye surface of the eye than conventional ophthalmological solutions. Hence maintaining a protective film on the eye surface is ensured, the eye is protected more effectively from external influences.

In cooperation of sodium hyaluronate and ectoin, the eye is supplied in addition with an intensive, long-term adhering moisture film and is protected from evaporation of the tears. Hence environmental- and dryness-caused irritations which lead to inflammatory symptoms are soothed just as are the typical symptoms of itchiness and burning which occur during allergic reactions.

The composition according to the invention is suitable in particular for the treatment or prophylaxis of dry eye (sicca syndrome), for the treatment or prophylaxis of inflammation of the conjunctiva (conjunctivitis) and/or for the treatment or prophylaxis of allergic reactions of the eye, such as e.g. hayfever.

Furthermore, the composition according to the invention protects the eye from premature cell damage. It is already known from the literature that, under hyperosmolar conditions, i.e. osmotic stress, the result is very rapid formation of reactive oxygen species (ROS). This was shown for example in primary human cornea epithelial cells (Ruzhi Deng, Xia Hua, Jin Li, Wei Chi, Zongduan Zhang, Fan Lu, Lili Zhang, Stephen C. Pflugfelder, and De-Quan Li, Oxidative Stress Markers Induced by Hyperosmolarity in Primary Human Corneal Epithelial Cells, PLoS One. 2015; 10(5): e0126561). These reactive molecules which, irrespective of the presence of an inflammation, are formed as reaction to osmotic stress, lead to cell damage (lipid peroxidation, oxidative change of proteins and oxidative DNA damage) as far as apoptosis.

The combination of hyaluronic acid and ectoin in the form of viscous eyedrops acts on various levels, resisting both the production of such reactive molecules and limiting damage if ROS are produced.

On the one hand, ectoin has a protein-stabilising function. As a result, ectoin can also stabilise antioxidants in the tear film, such as e.g. Cu—Zn-SOD. On the other hand, ectoin is a highly kosmotropic substance which demonstrates a strong interaction with water. It promotes the formation of water molecules in clusters and increases the surface tension of the water, which counteracts evaporation and correspondingly reduces or prevents the osmotic stress. Since ectoin itself is however osmotically active, the osmotic stress on the eye should however be reduced, a minimal use of ectoin is desired. By means of the synergistic effect in combination with hyaluronic acid, it is ensured that the advantageous properties of ectoin on the water-binding capacity and viscosity with a low ectoin concentration achieve a maximum effect. Finally, the synergistic viscosity increase imparts, by the combination of an ectoin- and a hyaluronic acid component, a longer dwell time and hence an extended protection since prolonged moistening of the eye surface reduces the osmotic stress and hence reduces the production of ROS.

It is hereby further preferred that the content of hyaluronic acid and/or an ophthalmologically acceptable salt of hyaluronic acid is from 0.10 to 1.00% by weight, further preferably from 0.10 to 0.45% by weight, further preferably from 0.125 to 0.45% by weight, particularly preferably from 0.15 to 0.25% by weight, in particular 0.15 to 0.20% by weight.

Alternatively or additionally, it is likewise preferred if the content of ectoin or an ophthalmologically acceptable ectoin derivative is from 0.75 to 3.00% by weight, preferably from 1.00 to 3.00% by weight.

The ophthalmologically acceptable salt of hyaluronic acid is thereby selected preferably from the group consisting of sodium hyaluronate, potassium hyaluronate and also mixtures or combinations hereof.

The ectoin is hereby in particular L-ectoin ((S)-2-methyl-1,4,5,6-tetrahydropyrimidine-4-carboxylic acid). Preferred ectoin derivatives are thereby selected from the group consisting of hydroxyectoin ((4S,5S)-5-hydroxy-2-methyl-1,4,5,6-tetrahydropyrimidine-4-carboxylic acid); salts, e.g. sodium- or potassium salts of ectoin; esters which can be obtained by conversion of the 4-carboxy group with alcohols, in particular straight-chain or branched mono- or bivalent alcohols with 1 to 20 carbon atoms, and/or of the 5-hydroxy group with carboxylic acids, in particular straight-chain or branched-chain mono- or bivalent alkyl carboxylic acids with 2 to 20 carbon atoms, e.g. alkylmonocarboxylic acids, and also acid addition salts with inorganic or organic acids.

It is hereby preferred in particular if the alkyl radicals of the alcohols or carboxylic acids have respectively up to 10 carbon atoms, in particular up to 5 carbon atoms.

According to a particularly preferred embodiment, the composition according to the invention is free of preservatives. There is thereby understood by preservative, according to the invention, any substance which can be used as ophthalmological preservative, such as e.g. the preservatives listed further on.

Preservatives can damage the precorneal tear film and lead to a reduction in the number of microvilli and microplicae of the surface corneal epithelium cells, which results in irritation and/or inflammation of the eye. By dispensing with preservatives in the solution according to the invention, such irritation and/or inflammation can hence be avoided.

In an alternative preferred embodiment, the composition according to the invention can likewise comprise one or more preservatives, in particular ophthalmologically usable or permitted preservatives. Those preservatives are preferred which are selected from the group consisting of quaternary ammonium compounds, such as for example benzalkonium chloride, cetrimide or polyquaternium 1; mercury compounds, such as for example thiomersal or phenylmercuric acetate; alcohols, such as for example chlorobutanol; carboxylic acids, such as for example sorbic acid; phenols, such as for example parabens; amidines, such as for example chlorohexidine; EDTA, in particular the disodium salt of EDTA; sodium hydroxymethylglucinate; sodium perborate; phosphonic acid; polydronium chloride; sodium chlorite and also mixtures or combinations hereof. However it is preferred that the composition is free of preservatives, in particular free of the previously mentioned preservatives.

Furthermore, it is advantageous that the ophthalmological composition comprises at least one buffer system, preferably a buffer system selected from the group consisting of borate buffer, citrate buffer, phosphate buffer, tris buffer, trometamol/maleic acid and also mixtures or combinations hereof.

In particular, it is hereby of advantage that the ophthalmological composition consists of ectoin or an ectoin derivative, hyaluronic acid or a hyaluronic acid derivative, a buffer system and water.

Likewise, the possibility is offered however that the ophthalmological composition comprises no buffer system.

A particularly preferred embodiment provides that the ophthalmological composition is free of phosphate. Phosphate-free, in the sense of the present invention, means that phosphate ions are contained, if at all, only below the detection limit of current analytical methods.

According to the invention there is understood by phosphate, any type of phosphate, i.e. e.g. also hydrogen phosphate, dihydrogen phosphate, diphosphate, triphosphate, polyphosphate and cyclophosphate. Within the scope of this preferred embodiment, this also has the result that the solution must comprise no phosphate buffer.

In the case of long-term applications of phosphate-containing solutions in the eye, the result can be cloudiness of the cornea due to incorporation and/or deposits of poorly soluble phosphates, such as e.g. calcium phosphate, which are incorporated or deposited in or on the cornea and also the conjunctiva of the eye. This degeneration of the cornea of the eye is also termed cornea band degeneration or band keratopathy. In fact small incorporations and/or deposits of poorly soluble phosphates in or on the cornea of the eye lead to hugely increased glare sensitivity which can be attributed to light scattering effected on the deposits or incorporations of poorly soluble phosphates. In particular, the visual capacity at night is consequently greatly impaired. As a result of the preferred complete absence of phosphate in the solution according to the invention, the formation of poorly soluble phosphates and the hence accompanying impairment in visual capacity because of cloudiness of the cornea can hence be avoided.

In a preferred embodiment, in particular in the presence of a buffer system, the osmolality (or the tonicity) of the solution is adjusted to 100-1,000 mOsm/kg, preferably 200-500 mOsm/kg, particularly preferably 220-350 mOsm/kg.

In a particularly preferred embodiment, the buffer is borate buffer.

It is hereby preferred in particular that the solution according to the invention comprises ectoin, sodium hyaluronate, borate buffer and water or consists hereof. Alternatively, it is likewise preferred that the solution according to the invention comprises ectoin, sodium hyaluronate, borate buffer, a preservative and water or consists hereof.

Furthermore, it is preferred that the borate buffer comprises boric acid and borax or consists of boric acid and borax.

In a further preferred embodiment of the solution according to the invention, the proportion of boric acid in the solution is 2.5 mg/ml to 10 mg/ml, preferably 7.2 mg/ml to 8.8 mg/ml, particularly preferably 7.7 mg/ml to 7.9 mg/ml, in particular 7.80 mg/ml to 7.82 mg/ml.

In a further preferred embodiment of the solution according to the invention, the proportion of borax in the solution is 0.1 mg/ml to 1 mg/ml, preferably 0.3 mg/ml to 0.7 mg/ml, particularly preferably 0.4 mg/ml to 0.5 mg/ml, in particular 0.41 mg/ml to 0.43 mg/ml.

Preferred pH values of the ophthalmological composition are hereby in the range of 5 to 9, preferably of 6 to 8, in particular of 6.8 to 7.8.

The ophthalmological composition according to the present invention preferably has a kinematic viscosity, measured by means of capillary viscosimetry as described in PhEur 7.2, General Methods 2.2.8, of 10 to 500 mm²/s, preferably 30 to 300 mm²/s, particularly preferably of 50 to 250 mm²/s.

In particular, the composition according to the invention is sterile.

Preferably the ophthalmological composition according to the present invention is configured in the form of eyedrops or an eye gel.

The ophthalmological composition can be applied 1 to 10 times daily, preferably 2 to 6 times daily in the eye. In particular, the ophthalmological composition is applied by dropping into the eye.

In a particularly preferred embodiment, the ophthalmological composition has the following formulation:

| | |
|---|---|
| sodium hyaluronate | 0.55 to 5 mg |
| ectoin | 0.50 to 3 mg |
| boric acid | 2.5 to 10 mg |
| borax | 0.1 to 1 mg |
| water ad | 1 ml |

The composition according to the invention with this formulation has a density of approx. 1.0068 g/cm³. The solution moistens cornea and conjunctiva and protects from excessive evaporation of tears. It can be used for prevention and treatment of an irritation and/or inflammation of the eye, the irritation and/or inflammation of the eye being caused by insufficient wetting of the eye with a tear film and/or by oversensitivity and/or allergy. The burning and itchy feeling in the eyes which is caused by irritation and/or inflammation disappears.

Furthermore, it is preferred that the irritation and/or inflammation of the eye is caused by insufficient wetting of the eye with a tear film and/or by oversensitivity and/or allergy.

The present invention is described in more detail with reference to the subsequent embodiments without restricting the present invention to the specially represented tests.

### Test Description:

In total 12 solutions with different concentrations of hyaluronic acid and ectoin were produced in a formulation base:
0. Production formulation base without hyaluronic acid and without ectoin:

Firstly, 781 mg boric acid and 42 mg borax are dissolved in succession in approx. 80 ml distilled water. After the raw materials are completely dissolved, the solution is filled up with distilled water to 100 ml.
1. Production formulation base without hyaluronic acid and with 1% ectoin:

1 g ectoin, 781 mg boric acid and 42 mg borax are dissolved in succession in approx. 965 ml distilled water. After the raw materials are completely dissolved, the solution is filled up with distilled water to 100 ml.
2. Production formulation base without hyaluronic acid and with 2% ectoin:

2 g ectoin, 781 mg boric acid and 42 mg borax are dissolved in succession in approx. 95 ml distilled water. After the raw materials are completely dissolved, the solution is filled up with distilled water to 100 ml.
3. Production formulation base without hyaluronic acid and with 3% ectoin:

3 g ectoin, 781 mg boric acid and 42 mg borax are dissolved in succession in approx. 95 ml distilled water. After the raw materials are completely dissolved, the solution is filled up with distilled water to 100 ml.
4. Production formulation base with 0.1% hyaluronic acid and without ectoin:

Firstly, approx. 45 ml distilled water is placed in a beaker and 100 mg hyaluronic acid is dissolved therein (solution 1). In a 2^{nd} beaker, 781 mg boric acid and 42 mg borax are dissolved in succession in approx. 45 ml distilled water (solution 2). After all the raw materials are completely dissolved, solution 2 is added slowly and with agitation to solution 1. Thereafter the solution is filled up with distilled water to 100 ml.
5. Production formulation base with 0.2% hyaluronic acid and without ectoin:

Firstly, approx. 45 ml distilled water is placed in a beaker and 200 mg hyaluronic acid is dissolved therein (solution 1). In a 2^{nd} beaker, 781 mg boric acid and 42 mg borax are dissolved in succession in approx. 45 ml distilled water (solution 2). After all the raw materials are completely dissolved, solution 2 is added slowly and with agitation to solution 1. Thereafter the solution is filled up with distilled water to 100 ml.
6. Production formulation base with 0.1% hyaluronic acid and 1% ectoin:

Firstly, approx. 45 ml distilled water is placed in a beaker and 100 mg hyaluronic acid is dissolved therein (solution 1). In a 2^{nd} beaker, 1 g ectoin, 781 mg boric acid and 42 mg borax are dissolved in succession in approx. 45 ml distilled water (solution 2). After all the raw materials are completely dissolved, solution 2 is added slowly and with agitation to solution 1. Thereafter the solution is filled up with distilled water to 100 ml.
7. Production formulation base with 0.1% hyaluronic acid and 2% ectoin:

Firstly, approx. 45 ml distilled water is placed in a beaker and 100 mg hyaluronic acid is dissolved therein (solution 1). In a 2^{nd} beaker, 2 g ectoin, 781 mg boric acid and 42 mg borax are dissolved in succession in approx. 45 ml distilled water (solution 2). After all the raw materials are completely dissolved, solution 2 is added slowly and with agitation to solution 1. Thereafter the solution is filled up with distilled water to 100 ml.
8. Production formulation base with 0.1% hyaluronic acid and 3% ectoin:

Firstly, approx. 45 ml distilled water is placed in a beaker and 100 mg hyaluronic acid is dissolved therein (solution 1). In a 2^{nd} beaker, 3 g ectoin, 781 mg boric acid and 42 mg borax are dissolved in succession in approx. 45 ml distilled water (solution 2). After all the raw materials are completely dissolved, solution 2 is added slowly and with agitation to solution 1. Thereafter the solution is filled up with distilled water to 100 ml.
9. Production formulation base with 0.2% hyaluronic acid and 1% ectoin:

Firstly, approx. 45 ml distilled water is placed in a beaker and 200 mg hyaluronic acid is dissolved therein (solution 1). In a 2^{nd} beaker, 1 g ectoin, 781 mg boric acid and 42 mg borax are dissolved in succession in approx. 45 ml distilled water (solution 2). After all the raw materials are completely dissolved, solution 2 is added slowly and with agitation to solution 1. Thereafter the solution is filled up with distilled water to 100 ml.
10. Production formulation base with 0.2% hyaluronic acid and 2% ectoin:

Firstly, approx. 45 ml distilled water is placed in a beaker and 200 mg hyaluronic acid is dissolved therein (solution 1). In a 2^{nd} beaker, 2 g ectoin, 781 mg boric acid and 42 mg borax are dissolved in succession in approx. 45 ml distilled water (solution 2). After all the raw materials are completely dissolved, solution 2 is added slowly and with agitation to solution 1. Thereafter the solution is filled up with distilled water to 100 ml.
11. Production formulation base with 0.2% hyaluronic acid and 3% ectoin:

Firstly, approx. 45 ml distilled water is placed in a beaker and 200 mg hyaluronic acid is dissolved therein (solution 1). In a 2^{nd} beaker, 3 g ectoin, 781 mg boric acid and 42 mg borax are dissolved in succession in approx. 45 ml distilled water (solution 2). After all the raw materials are completely dissolved, solution 2 is added slowly and with agitation to solution 1. Thereafter the solution is filled up with distilled water to 100 ml.

The solutions produced as previously were tested for their viscosity.

The viscosity measurements were implemented with an Ubbelohde viscosimeter 501 20/II as described in PhEur 7.2, General Methods 2.2.8.

For the individual compositions, the following viscosity values were thereby obtained, which values are reproduced in the subsequent table:

| | | Viscosity, |
|---|---|---|
| | | measurement with |
| | | capillary II (mm²/s) |
| 0* | Formulation base | 1.2 |
| | without HA and without ectoin | |
| 1* | Formulation base | 1.2 |
| | without HA and 1% ectoin | |
| 2* | Formulation base | 1.3 |
| | without HA and 2% ectoin | |
| 3* | Formulation base | 5.8 |
| | without HA and 3% ectoin | |
| 4* | Formulation base | 18.3 |
| | without ectoin and 0.1% HA | |
| 5* | Formulation base | 93.0 |
| | without ectoin and 0.2% HA | |
| 6 | Formulation base | 20.8 |
| | 0.1% HA and 1% ectoin | |
| 7 | Formulation base | 21.4 |
| | 0.1% HA and 2% ectoin | |
| 8 | Formulation base | 22.4 |
| | 0.1% HA and 3% ectoin | |
| 9 | Formulation base | 144.1 |
| | 0.2% HA and 1% ectoin | |
| 10 | Formulation base | 146.7 |
| | 0.2% HA and 2% ectoin | |
| 11 | Formulation base | 158.6 |
| | 0.2% HA and 3% ectoin | |

*Comparative tests

As is evident from tests 6 to 8 or 9 to 11 according to the invention, a clear, synergistic influence on the viscosity in the presence of hyaluronic acid and ectoin takes place, the viscosities in these combined tests turning out to be higher than the sum of the individual measured values in the individual tests (tests 1 to 3 or tests 4 to 5).

In addition, the water-binding capacity of seven different mixtures (no. 12-18) of hyaluronic acid and ectoin was determined with different weight ratios HA:ectoin. Two different methods were applied for this purpose.

On the one hand, the water-binding capacity was determined with the help of a gravimetric method. The proportions m_{HA} and m_{Ec} of hyaluronic acid and ectoin, indicated in the subsequent table, were placed in an Eppendorf vessel, the proportions relating to a total weight (hyaluronic acid+ectoin) of 100 mg. The total mass of the filled Eppendorf vessel was noted. Subsequently, water was added in drops until a small water excess was present and a clear solution had been produced. This solution was subjected to centrifugation at 200 rpm. The supernatant water was thereby removed and the total weight of the Eppendorf vessel was determined again. The water-binding capacity was then calculated as the difference in the total weight after centrifugation and the initially noted total weight.

The subsequent table shows the results of the gravimetric determination of the percentage water-binding capacity (WBK). In addition, the table shows the values for the water-binding capacity which would have been expected in the case of a purely additive effect on the basis of the WBK for pure hyaluronic acid (test 12) and of the WBK for pure ectoin (test 18). The deviation of the measured WKB from the calculated theoretical WBK reflects the synergistic effect.

| | | | WBK | WBK | Synergistic |
|---|---|---|---|---|---|
| | m_{HA} | m_{Ec} | (gravimetric) | calculated | effect |
| Mixture | in % | in % | in % | in % | in % |
| 12 | 100 | 0 | 32.00 | — | — |
| 13 | 20 | 80 | 45.91 | 43.0 | 6.8 |
| 14 | 40 | 60 | 70.04 | 54.0 | 29.7 |
| 15 | 50 | 50 | 74.00 | 59.5 | 24.4 |
| 16 | 60 | 40 | 74.80 | 65.0 | 15.1 |
| 17 | 80 | 20 | 79.20 | 76.0 | 4.2 |
| 18 | 0 | 100 | 87.00 | — | — |

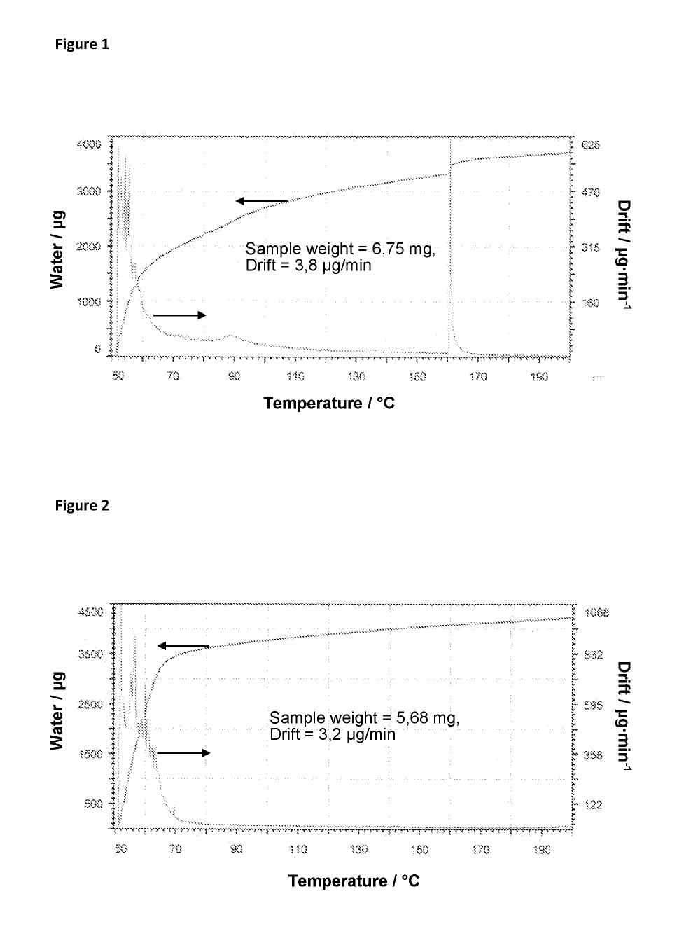

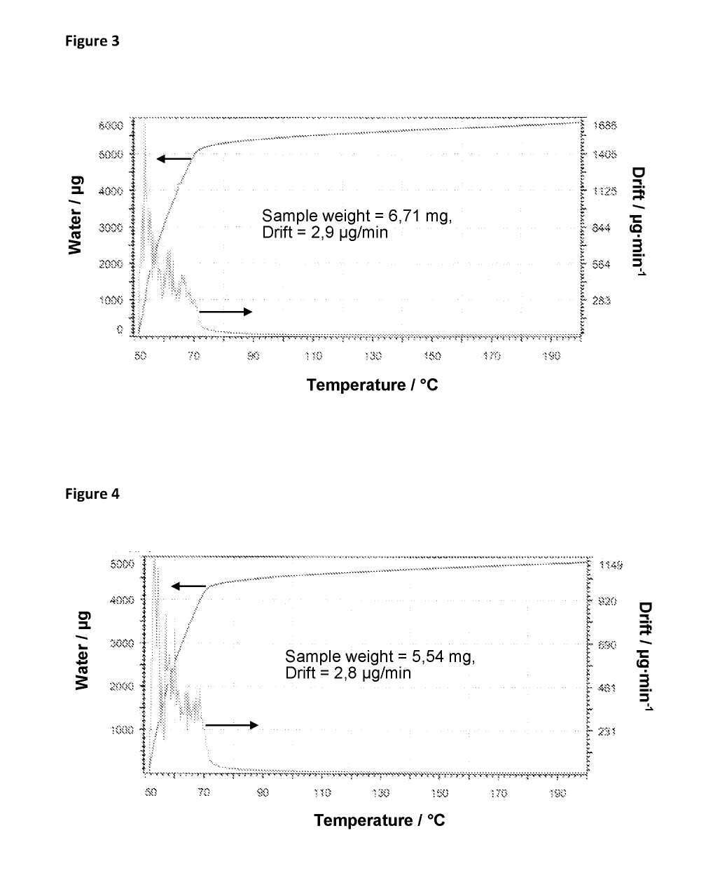

On the other hand, the water-binding capacity was determined by means of the indirect Karl Fischer Titration with oven technology. For this purpose, a sample of 5-7 mg was removed from the Eppendorf vessel after centrifugation and withdrawal of the supernatant water in the gravimetric method. This sample was heated to an initial temperature of 50° C. and subsequently heated in a closed vessel with a heating rate of 2° C./min to 200° C. By means of gas rinsing, the thereby evaporating water was conducted via a hollow needle into a titration cell. The water collected there reacted with a Karl Fischer solution and the water content of the sample and the water-binding capacity were calculated via the end point of the titration curve. FIGS. 1-4 show the thermograms which were plotted in the above-described Karl Fischer analysis of the mixtures 13, 14, 16 and 17: FIG. 1 shows the measured curves which were obtained during analysis of mixture 13. FIG. 2 shows the thermograms of the analysis of mixture 14. FIG. 3 shows the thermograms obtained during analysis of mixture 16 and FIG. 4 the results of the analysis of mixture 17.

From these Figures, the sample weight and also the detected mass of water at 200° C. can be read off. For correction of the determined mass of water, in addition the temporal drift of the measuring apparatus must be included.

The subsequent table combines the results of determination of the percentage water-binding capacity (WBK) according to the indirect Karl Fischer method with oven technology. In addition, the table shows the values for the water-binding capacity which would have been expected with a purely additive effect on the basis of the WBK for pure hyaluronic acid (test 12) and the WBK for pure ectoin (test 18). The deviation of the measured WKB from this theoretically calculated WBK also forms here a measure of the synergistic effect.

| | | | WBK | | |
|---|---|---|---|---|---|
| | | | (according to | WBK | Synergistic |
| | m_{HA} | m_{Ec} | Karl Fischer) | calculated | effect |
| Mixture | in % | in % | in % | in % | in % |
| 12 | 100 | 0 | 31.80 | — | — |
| 13 | 20 | 80 | 50.60 | 44.2 | 14.5 |
| 14 | 40 | 60 | 64.30 | 56.6 | 13.6 |
| 15 | 50 | 50 | 80.20 | 62.8 | 27.7 |
| 16 | 60 | 40 | 83.65 | 69.0 | 21.2 |
| 17 | 80 | 20 | 83.95 | 81.4 | 3.1 |
| 18 | 0 | 100 | 93.80 | — | — |

The results of the determination of the WBK verify that—irrespective of the measuring method—there is a synergistic effect of hyaluronic acid and ectoin on the water-binding capacity. In the case of a purely additive effect, for example in mixture 15 a value of 62.8% (=0.5·32.00+0.5·87.00) for water-binding capacity would have been expected by means of Karl Fischer Titration, however a value of 80.2% was measured. The same applies for the gravimetric determination: with a purely additive effect, for mixture 15 a water-binding capacity of 59.5% (=0.5·31.80+0.5·93.80) would have been expected, however a WBK of 74.0% was measured.

1. An ophthalmological composition, comprising:
0.055 to 2.00% by weight of hyaluronic acid, an ophthalmologically acceptable salt of hyaluronic acid, or a mixture thereof,
0.60 to 5.00% by weight of ectoin or an ophthalmologically acceptable ectoin derivative, and
ad 100% by weight of water,
wherein the composition is free of a further pharmaceutically active ingredient.

2. The ophthalmological composition according to claim 1, for the treatment or prophylaxis of dry eye, for the treatment or prophylaxis of inflammation of the conjunctiva, for the treatment or prophylaxis of allergic reactions of the eye, or a combination thereof.

3. The ophthalmological composition according to claim 1, wherein the content of hyaluronic acid, an ophthalmologically acceptable salt of hyaluronic acid, or a mixture thereof is from 0.10 to 1.00% by weight of the composition and the ectoin or an ophthalmologically acceptable ectoin derivative is from 0.75 to 3.00% by weight, of the composition.

4. The ophthalmological composition according to claim 1, wherein the ophthalmologically acceptable salt of hyaluronic acid is selected from the group consisting of sodium hyaluronate, potassium hyaluronate and mixtures or combinations thereof.

5. The ophthalmological composition according to claim 1, wherein the ectoin is L-ectoin, hydroxyectoin, a salt, an ester and a mixture thereof.

6. The ophthalmological composition according to claim 5, wherein the alkyl radical of the alcohol or carboxylic acid have respectively up to 10 carbon atoms.

7. The ophthalmological composition according to claim 1, wherein the composition is free of preservatives or comprises at least one preservative.

8. The ophthalmological composition according to claim 1, wherein the composition comprises at least one buffer system selected from the group consisting of borate buffer, citrate buffer, phosphate buffer, tris buffer, trometamol/maleic acid and mixtures or combinations thereof
or comprises no buffer system.

9. The ophthalmological composition according to claim 1, wherein the composition is free of phosphate ions.

10. The ophthalmological composition according to claim 1, wherein the osmolality of the solution is 100-1,000 mOsm/kg.

11. The ophthalmological composition according to claim 1, wherein the composition has a pH value of 5 to 9.

12. The ophthalmological composition according to claim 1, wherein the composition has a kinematic viscosity, measured by capillary viscosimetry according to PhEur 7.2, General Methods 2.2.8, of 10 to 500 mm²/s.

13. The ophthalmological composition according to claim 1, wherein the composition is sterile.

14. The ophthalmological composition according to claim 1, wherein the composition is configured in the form of eyedrops or an eye gel.

15. The ophthalmological composition according to claim 1, wherein the composition is adapted to be applied one to 10 times daily by dropping.

16. The ophthalmological composition according to claim 5, wherein the salt is chosen from a sodium salt of ectoin, a potassium salt of ectoin.

17. The ophthalmological composition according to claim 7, wherein the preservative is chosen from quaternary ammonium compound, a mercury compound, an alcohol, a carboxylic acid, a phenol, an amidine, an EDTA, sodium hydroxymethylglucinate; sodium perborate; phosphonic acid; polydronium chloride; sodium chlorite and also mixtures or combinations thereof.

18. The ophthalmological composition according to claim 3, wherein the hyaluronic acid, an ophthalmologically acceptable salt of hyaluronic acid, or a mixture thereof is from 0.15 to 0.20% by weight and/or of the composition and the ectoin or an ophthalmologically acceptable ectoin derivative is from 1.00 to 3.00% by weight of the composition.

19. The ophthalmological composition according to claim 5, wherein the ester is a reaction product obtained by conversion of a 4-carboxy group with an alcohol.

20. The ophthalmological composition according to claim 19, wherein the alcohol comprises 1 to 20 carbon atoms.
